(19) United States
(12) Patent Application Publication
(10) Pub. No.: US 2014/0005260 A1
(43) Pub. Date: Jan. 2, 2014

(54) METHOD FOR INHIBITING CANCER METASTASIS BY AMIODARONE

(71) Applicants: Huai-Jen Tsai, Taipei City (TW); Ming-Shen Dai, Taipei City (TW); Ying-Shin Chen, Taipei City (TW); Hung-Chieh Lee, New Taipei City (TW); Hao-Chan Lo, Taipei City (TW); Mei-Yan Su, Taipei City (TW)

(72) Inventors: Huai-Jen Tsai, Taipei City (TW); Ming-Shen Dai, Taipei City (TW); Ying-Shin Chen, Taipei City (TW); Hung-Chieh Lee, New Taipei City (TW); Hao-Chan Lo, Taipei City (TW); Mei-Yan Su, Taipei City (TW)

(73) Assignees: TRI-SERVICE GENERAL HOSPITAL, Taipei City (TW); NATIONAL TAIWAN UNIVERSITY, Taipei City (TW)

(21) Appl. No.: 13/730,599

(22) Filed: Dec. 28, 2012

(30) Foreign Application Priority Data
Jun. 28, 2012 (TW) 101123339

(51) Int. Cl. A61K 31/343

(52) U.S. Cl. CPC A61K 31/343
USPC 514/469

(57) ABSTRACT

Amiodarone inhibits the invagination during zebrafish heart development and makes the defect on valves development. The present invention demonstrates that Amiodarone inhibits cancer metastasis and provides a method for inhibiting cancer metastasis in a subject in need thereof comprising administering to the subject a pharmaceutically effective amount of an Amiodarone or its salt.

### FIELD OF THE INVENTION

The present invention is related to a method for inhibiting cancer metastasis by Amiodarone or its salt.

### BACKGROUND OF THE INVENTION

Amiodarone is categorized as a type III antiarrhythmic drug. It is considered a broad-spectrum antiarrhythmic agent because it has multiple and complex effects on the electrical activity of the heart. As such, Amiodarone is effective in treating tachyarrhymias, including re-entry supraventricular tachycardias, ventricular tachycardia, atrial arrhythmias and ventricular fibrillation. While Amiodarone is considered the antiarrhythmic treatment of choice, it is classified as a category D drug. Consequently, caution should be exercised before using Amiodarone for pregnant women, as it causes embryonic hypothyroidism and hyperthyroidism. In contrast, it is showed that long-term use of Amiodarone has no effect on embryogenesis.

In previous study, no developmental effects, except one case of hypothyroidism, was demonstrated. However, these studies focused on embryos older than 6 months when most organs, including the heart, have been completely formed. There is no epidemiological data for women who were taking Amiodarone when they inadvertently became pregnant. Still, based on the evidence at hand, it is important to know whether Amiodarone could be toxic for embryos at an early developmental stage because the half-life of Amiodarone is reported to be 26-107 days, and Amiodarone could be prescribed in the case of undetected pregnancy or pregnancy within the gestational period.

To study the toxicity of Amiodarone on heart development, zebrafish was used as a system model since the transparency of the embryos allows us to directly observe cardiac development without invasive procedures. As such, zebrafish is an excellent organism to study cardiovascular genetics and defects (Nat Rev Genet. 2001; 2:39-48). Two zebrafish heart-specific fluorescence transgenic lines are available for the in vivo study of cardiac development: Tg(cmlc2:EGFP) with heart-specific green fluorescence (Dev Dyn 2003; 228: 30-40) and Tg(cmlc2:HcRFP) with heart-specific infrared emission (J Struct Biol 2004; 147: 19-30; Opt Lett 2006; 31(7): 930-2). In addition, early-stage cardiac development of zebrafish is similar to that of human in many respects, such as the migration of cardiac precursor cells towards the central line, heart tube formation, early chamber formation and the looping process.

The muscular walls of the heart consist of three major “layers.” The bulk of the walls is made up of a layer of cardiac muscle and is called the myocardium. The muscle is enclosed on the outside by the epicardium and on the inside by the endocardium. The heart is also covered completely by a protective sac called the pericardium. There are 5 stages of heart development: specification of cardiac precursor cells, migration of cardiac precursor cells and fusion of the primordial, heart looping, heart chamber formation and septation and valve formation.

The heart tube is composed of an outer layer of myocardium and an inner lining of endocardial cells, separated by an extensive extracellular matrix (ECM) referred to as cardiac jelly. After rightward looping of the heart, the cardiac jelly overlying the future atrioventricular canal (AVC) and outflow tract (OT) expands into swellings known as cardiac cushions. Cardiac valves, such as the atrioventricular valves and semilunar valves are differentiated from the endocardium. In the development of mammalian cardiac valve, the endocardial cells are regulated by several signaling pathways including Wnt/β-catenin, Notch, Vascular Endothelial Growth Factor (VEGF) and BMP/TGF-β from the myocardium and the extracellular matrix from the cardiac jelly such as Versican and hyaluronic acid. These signaling regulate the proliferation and specification of the endocardial cells. If the signaling is interfered, it will lead to the cardiac valve defects. Next, the endocardial cells which do not have the migratory ability transform to the messenchymal cells which have the migratory ability; the process is called epithelial-mesenchymal transition (EMT). The formation of the cardiac cushions is a complex event characterized by endothelial-mesenchymal transition (EndMT) of a subset of endothelial cells that are specified in the cushion-forming regions to delaminate and invade the cardiac jelly, where they subsequently proliferate and complete their differentiation into mesenchymal cells.

The development of zebrafish heart begins at 19.5 hours post-fertilization (hpf). At this time, cardiac precursor cells are moving toward the embryonic midline and the cardiac crescent is formed. At 22 hpf, the heart tube is developed and the first heart beat begins. The heart looping begins at 36 hpf and the early chamber is formed. The endothelial cells in the atrioventricular canal begin valve specification and until 55 hpf, the endothelial cells in the AVC proceed invagination. Unlike in mice or chickens, zebrafish atrioventricular endocardial cells do not appear to give rise to mesenchymal cushions. The AVC endocardium extends processes into the space between the endocardium and myocardium and invaginates, beginning at the ventricular side of the AVC, to directly form a valve leaflet. Now this process is termed invagination (Development 2008; 135:1179-1187). At 60 hpf, endocardial cushion is developed; and at 96 hpf, the valve begins remolding and the heart is gradually developed.

### BRIEF DESCRIPTION OF THE DRAWINGS

FIG. 1 shows the in vivo observation of the cardiac valve defects in the Amiodarone treated zebrafish embryos. HGM/2 PF was applied to observe the valve development of transgenic zebrafish Tg(cmlc2:HcRFP) in vivo. RFP marked the myocardial cells, while third harmonic generation marked all cells. Second harmonic generation marked the cardiac muscles. Valve development in zebrafish started around 36-40 hpf.

FIG. 2 shows the expression pattern of Snail family during zebrafish embryogenesis. A-D are lateral views of 72 hpf embryos. C′ and D′ are ventral views of cardiac field. WISH reveals that snai1a is not detected at the heart field (A). snai1b is detected at the atrioventricular (AV) canal (C′ red arrow). Incubation of 15 μM Amiodarone from 55 to 72 hpf did not influence the snail1a and snail1b at head reagen, but the expression of snai1b at AV canal were lost (D′ red arrow). V: ventricle, A: atrium.

FIG. 3 shows that zebrafish embryos treated with Amiodarone or knockdown of snai1b expression increase Cdh5 protein level. 72 hpf Zebrafish embryos which were treated with 15 μM Amiodarone from 55 to 72 hpf (lane2) or injected with 1.5 ng snail1b-MO (SEQ ID NO: 3) at one cell staged (lane 3) were analyzed with the Cdh5 level. GAPDH (Glyceraldehyde 3-phosphate dehydrogenase) were used as the internal control.

FIG. 4 shows the expression patterns of versican family during zebrafish embryogenesis. WISH of vcana, vcanb and s-vcanb during 72 hpf embryos are shown. A-F are ventral views of cardiac field. A′-F′ are lateral views of head region.

FIG. 5 shows that S-vcanb is involved in the inhibition of cardiac valve formation of Amiodarone. WISH of snail1b (A-D, I-L) and cdh5 (E-H, M-P) in WT (A, E, I, M), Amiodarone treated embryos (B, F, J, N), vcana-morphants (C, G), s-vcanb-morphants (K, O), Amiodarone treated vcana-morphants (D, H) and Amiodarone treated s-vcanb-morphants (L, P) are shown.

FIG. 6 shows that Amiodarone influences S-versican-b/Snail1b/Cdh5 signaling through the EGF motif of S-versican-b. (A) Diagram illustrating the mutation site of the s-versican-b at the EGF motif. 6 primers (L1-3 and R1-3) containing 8 mutation site (G→C), which destroy the connection of EGF motif, were used to generate the pS-vcanbmE. WISH of s-vcanb (B-E), snail1b (F-I) and cdh5 (J-M) in embryos treated with Amiodarone (C, G, K) or over-expressed mutant form of S-vcanbmE (D, H, L) or wild-type S-vcanb (E, I, M) are shown.

FIG. 7 shows that Amiodarone induced zebrafish S-vcanb repress EGFR signaling in the heart field. WISH of snail1b (A, B) and cdh5 (C, D) in control (A, C) and AG1478 (B, D) treated embryos are shown. EGFR targeting antibodies were used to depict EGFR monomers and dimers in 72 hpf WT and Amiodarone treated embryos (E). Amiodarone treatment repressed the Phosphorylation of EGFR on Tyr-845 (F). Knockdown of s-vcanb (SEQ ID NO: 2) rescued the reduced EGFR phosphorylation on Tyr-845 in Amiodarone treated embryos (G). GSK3β activity was increased in Amiodarone treated embryos, and was rescued by knockdown of s-vcanb but not vcan-a (H).

FIG. 8 shows that 10˜30 μM Amiodarone does not influence B16OVA and JC survival. The cell survival assay to measure drug-induced cytotoxicity was examined by MTT following 24 hr treatment of 10 to 50 μM Amiodarone. Data is represented as a percentage of the DMSO control (DMSO), which is set to 100% and is expressed as mean±SEM (n=3).

FIG. 9 shows that Amiodarone induces Versican V2 expression and reduces Snail but increases E-cadherin protein level. B16OVA (A), JC(B) and 4T-1 cells (C) were treated with 15 μM Amiodarone for 24 hr and Versican V1(Vcan V1), V2 (Vcan V2), Snail and E-cadherin protein level were analyzed.

FIG. 10 shows that Amiodarone inhibits B16OVA cell migration. Confluent monolayer of B16OVA cells were mechanically wounded with a pipette tip and photos were obtained at 0 h, 24 h and 48 h after stimulation of 10, 15, 20 and 30 μM of Amiodarone (A). B shows the quantification of wound healing assay in 3 independent clones.

FIG. 11 shows that Amiodarone inhibits JC cell migration. Confluent monolayer of JC cells were mechanically wounded with a pipette tip and photos were obtained at 0 h, 24 h and 48 h after stimulation of 10, 15, 20 and 30 μM of Amiodarone (A). B shows the quantification of wound healing assay in 3 independent clones.

FIG. 12 shows that Amiodarone inhibits 4T-1 cell migration. Confluent monolayer of 4T-1 cells were mechanically wounded with a pipette tip and photos were obtained at 0 h, 24 h and 48 h after stimulation of 10, 15 and 20 μM of Amiodarone.

FIG. 13 shows that Amiodarone inhibits cell migration. Confluent monolayer of B16OVA (A) and JC cells (B) were mechanically wounded with a pipette tip and photos were obtained at 0 h, 24 h and 48 h after stimulation of 15 μM of Amiodarone or Mitomycin C.

FIG. 14 shows the morphological changes in Amiodarone treated cells. Wt and Amiodarone treated cells at a growing phase are shown. Amiodarone treated B16OVA cells appeared striper and more spread out than control cells.

FIG. 15 shows that Amiodarone inhibits cell proliferation through inhibiting EGFR downstream PI3K/AKT and ERK signaling. B16OVA (A), JC(B) and 4T-1 cells (C) were treated with 15 μM Amiodarone and the level of AKT, pAKT, ERK and p-ERK were analyzed. The total level of AKT and ERK appeared unaffected in Amiodarone treated cells, the level of pAKT and pERK42/44 were reduced greatly in Amiodarone treated cells. β-actin was used as internal control.

FIG. 16 shows the tumor growth in Balb/c mice after injection of Amiodarone or mitomycin C treated cells. Tumors derived from the injection of JC cells were treated with Amiodarone or mitomycin C. =untreated tumor; [symbol] =Mitomycin C treated tumors; and ▴=Amiodarone treated tumors. For each group of tumors, the average volume for 3 tumors is plotted. Bars=95% confidence intervals.

### SUMMARY OF THE INVENTION

The present invention provides a method for inhibiting cancer metastasis in a subject in need thereof comprising administering to the subject a pharmaceutically effective amount of an Amiodarone or its salt.

### DETAIL DESCRIPTION OF THE INVENTION

Unless otherwise specified, “a” or “an” means “one or more”.

In the normal embryonic development, some epithlial cells are undergoing epithelial-mesenchymal transition (EMT) for migration. In some pathological conditions such as carcinogenesis, the EMT is being restarted. Resent studies show that EMT plays an important role for the connection between normal cells and transformed stem cells. During the process of epithelial-mesenchymal transition, some epithlial cells are losing their typical cone shapes and the cell-cell connection and reconstructing the new cytoskeleton. Besides, these cells are losing the adhesion ability, down-regulating the E-cadherin, up-regulating proteins such as Vimentin, Fibronectin, N-cadherin and increasing cell motility.

The “Amiodarone” used herein comprises but not limits to Amiodarone or its pharmaceutically acceptable isomers, salts or compounds that have the same effect as Amiodarone when administering to the subject. The “Amiodarone” can further comprise its pharmaceutically acceptable carriers, adjuvants or excipient.

Amiodarone is a common antiarrhythmia agent, but its effects on the embryonic development are still unknown. In the present invention, zebrafish is used as a model for understanding the effects of Amiodarone on the heart development. Based on this model, the present invention demonstrates that Amiodarone inhibits the cardiac valve invagination during zebrafish heart development and makes the defect on valves development. Although cancer cells can also regulate the EMT to gain the ability of motility and metastasis, the EMT mechanism in cancer cells is different from it in the heart development. The present invention also demonstrates that Amiodarone inhibits cancer metastasis.

Therefore, the present invention provides a method for inhibiting cancer metastasis in a subject in need thereof comprising administering to the subject a pharmaceutically effective amount of an Amiodarone or its salt.

In the preferred embodiment of the present invention, the salt is hydrochloride and the cancer is selected from cancer cells with an abnormal expression of Versican. In the more preferred embodiment of the present invention, the cancer is selected from cancer cells with an abnormal expression of EGFR.

In the preferred embodiment of the present invention, the cancer is selected from melanoma, mammary adenocarcinoma, lung cancer, ovarian cancer or cervical cancer.

Based on the present invention, the inhibition of cancer metastasis is due to an inhibition of epithelial-mesenchymal transition of the cancer by the Amiodarone which is through an inhibition of EGFR signaling pathway by the Amiodarone to inhibit an expression of Snail and promotes an expression of E-cadherin.

Based on the present invention, the subject is selected from but not limited to a mammal or a human.

The present invention further provides a method for inhibiting cancer proliferation in a subject in need thereof comprising administering to the subject a pharmaceutically effective amount of an Amiodarone or its salt.

In the preferred embodiment of the present invention, the salt is hydrochloride and the cancer is selected from cancer cells with an abnormal expression of Versican. In the more preferred embodiment of the present invention, the cancer is selected from cancer cells with an abnormal expression of EGFR.

In the preferred embodiment of the present invention, the cancer is selected from melanoma, mammary adenocarcinoma, lung cancer, ovarian cancer or cervical cancer.

Based on the present invention, the inhibition of cancer proliferation is due to an inhibition of EGFR signaling pathway by the Amiodarone to inhibit PI3K/AKT and ERK signaling pathway.

Based on the present invention, the subject is selected from but not limited to a mammal or a human.

### EXAMPLES

The examples below are non-limiting and are merely representative of various aspects and features of the present invention.

Observation of Zebrafish Transgenic Lines and Heart Development

The zebrafish AB strain, as well as transgenic lines Tg(cmlc2:HcRFP) and Tg(cmlc2:EGFP), were cultured as previously described. Heart formation was observed under fluorescent stereomicroscopy (MZ Leica). Valve formation was observed in vivo by using Harmonic Generation Microscopy assisted by Two-Photon Fluorescence Microscopy (Opt Lett, 2006. 31(7), 930-932). The excitation source was a femtosecond Cr:forsterite laser with an output wavelength of 1230 nm. Second harmonic generation (SHG) and third harmonic generation (THG; J Struct Biol. 2004 147: 19-30) were applied to observe valve development in transgenic zebrafish Tg(cmlc2:HcRFP) in vivo. RFP marked the myocardial cells, while THG (410 nm) marked all cells in yellow, and SHG (615 nm) marked the skeletal and cardiac muscles in green. Paraffin sectioning with Hematoxylin and Eosin (H&E) staining was also used to perform histochemical analysis of the heart.

Drug Treatment with Zebrafish Embryos

EGFR inhibitor AG 1478 (CalBiochem) was dissolved in DMSO and stocked as 1 mM at −20° C. Working concentration used in this study was 7 μM; Amiodarone (Sigma) was dissolved in water at 65° C. for 2 h and stocked as 900 μM at 4° C. Before use, the solution was re-dissolved at 65° C. for 1 h. In the control group, 100 embryos were placed in a 9 cm dish filled with a volume of 30 ml embryo medium containing 0.2 mM 1-phenyl-2-thio-urea (Sigma). In the experimental group, the protocol was identical to the control group, except that embryos at different stages were treated with concentrations of Amiodarone that ranged from 3 to 30 μM, and embryos were exposed to treatment from 12 to 84 h. Long-term treatment during 12-72 hpf included the specification stage of valve formation at 36-55 hpf and the invagination stage of valve formation at 55 hpf. Treatment during 12-48 hpf was used to examine the gene markers versican and cdh5 which expressed at the AVC. During treatment, Amiodarone was refreshed every 24 h, and after treatment, embryos were washed twice with embryo medium, collected into a new 9 cm dish, and then incubated at 28° C.

### Example 1

### Cardiac Valves of Zebrafish were Directly Observed In Vivo

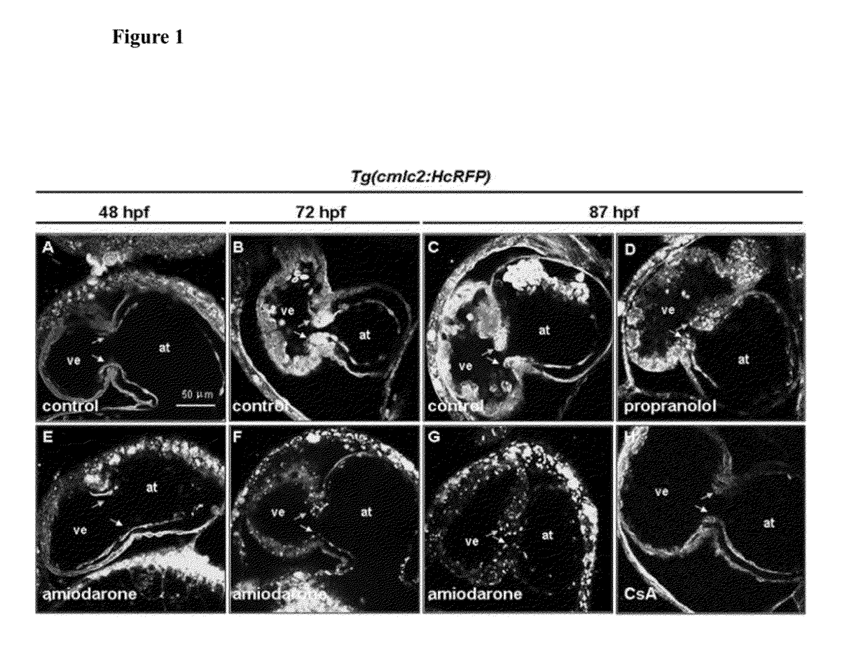

By using HGH/2 PF to examine the zebrafish embryos derived from transgenic line Tg(cmlc2:HcRFP), the dynamics of valve development in vivo could easily be observed. For example, at 48 hpf, there was a single layer of cells in the endocardium at the AVC (FIG. 1A). At 72 hpf, a bulged structure was observed at the AVC (FIG. 1B), and the endocardial cells continued to move towards cardiac jelly and gradually elongated to form the structure of valves at 96 hpf. Finally, at 87 hpf, the elongated structure was protruding towards the ventricle (FIG. 1C). However, if these embryos were treated with 3.00 μM CsA, a drug known to repress epithelial-mesenchymal transition (EMT) and thus cause valve defect (Nature 1998; 392:186-90) during 12-87 hpf, valves were not formed, which, again, was clearly observed at 87 hpf under HGH/2 PF (FIG. 1H). Interestingly, when zebrafish embryos were treated with 15 μM Amiodarone during 12-48 hpf, no difference between treated and untreated embryos was noted in endocardial cells at the AVC where only a single layer of cells were observed (FIG. 1A vs. E). However, when embryos were treated with the same dosage of Amiodarone during 12-72 hpf, only a small aggregation of cells were seen at the upper valve site and there were no valve structure at the lower site (FIG. 1B). Furthermore, compared to untreated embryos (FIG. 1C), embryos treated with Amiodarone during 12-87 hpf displayed almost no cellular aggregation at the valve region (FIG. 1G).

### Example 2

### The Expression Pattern of Snail Family During Zebrafish Embryogenesis

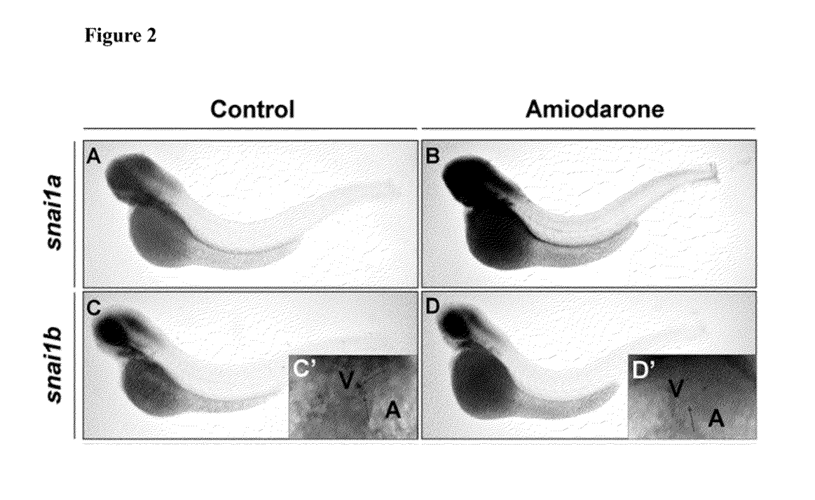

In the molecular mechanism, it was observed that the gene expression involved in epithelial-mesenchymal transition (EMT) during zebrafish embryogenesis was affected by Amiodarone treatment. During mouse embryonic valve formation, Snail (Snail) in Snail family acts as the receptor of cdh5 that inhibit the transcription of cdh5. To understand whether Amiodarone regulates snail gene in the heart, whole mount in situ hybridization (WISH) was used. WISH was performed as previously described (Nucleic Acids Res 2010; 38:4384-93). Riboprobe of cdh5 was prepared by cloning its partial DNA fragment, while riboprobe of versican was provided by Haramis (Nature 2003; 425:633-7). It was found that snai1a mRNA was not expressed in the heart of wild-type zebrafish at 72 hpf (FIG. 2A). Embryos treated with Amiodarone during 55-72 hpf were observed at 72 hpf. Although the expression of snail a mRNA increased in the head, it still was not expressed in the heart (FIG. 2B). snai1b mRNA was expressed at the AVC of the wild-type zebrafish at 72 hpf (FIG. 2C, C′ arrows), however after treating with Amiodarone, the expression of snai1b mRNA was decreased or even disappeared (FIG. 2D). It showed that snai1b of the Snail family involved in the mechanism of inhibiting heart valve formation of Amiodarone.

### Example 3

### Zebrafish Embryos Treated with Amiodarone or Knockdown of snai1b Expression Increases Cdh5 Protein Level

Cdh5 antibody was used to detect the amount of Cdh5 protein expression by Western blot. The embryos were dechorionated and deyolked with two extra washing steps as described in Link et al. (BMC Dev Biol 2006; 6:1). Deyolked samples were dissolved in 2 μl of 2×SDS sample buffer for each embryo and incubated for 5 min at 95□. After full-speed centrifugation for 1 min in a microcentrifuge to remove insoluble particles, total proteins extracted from embryos were analyzed on a 12% SDS-PAGE gel, and Western blot analysis was performed (J Biol Chem 2011; 286:6855-64) using antiserum against mouse Cdh5 (15; 1:10,000). Anti-α-tubulin and anti-β-actin served as a protein loading control. Knockdown experiments were performed as follows: morpholino nucleic acid oligomers (MOs) were purchased from GeneTools (USA): vcana-MO (AGGAAGATACCCATATTTCTGCTGA, SEQ ID NO: 1); s-vcanb-MO (CTGAAACACCCATGGGAGTGGACAT, SEQ ID NO: 2); snai1b-MO (TTGACAAGA AATGAGCGTGGCATCT, SEQ ID NO: 3) (Development 134, 4073-4081, 2007); cdh5-MO (TTTACAAGACCG TCTCCTTTCCAA, SEQ ID NO: 4) (Developmental Dynamics. 2004. 231, 204-213; PloS One 2010. 5, e8807); troponin T2a, cardiac-MO (5′-CATGTTTGCTCTGATCTGACACGCA-3′, SEQ ID NO: 5) (Nat. Genet. 2002. 31, 106-110); and standard control-MO (CCTCTTACC TCAGTTACAATTTATA, SEQ ID NO: 6). All MOs were prepared at a stock concentration of 1 mM and diluted to the desired concentration, specifically, 8, 12 and 16 ng for vcana-MO (SEQ ID NO: 1) and s-vcanb-MO (SEQ ID NO: 2); 4, 8, 12 and 16 ng for control-MO (SEQ ID NO: 6); and 0.8, 1.2, 1.6 and 2 ng for snail1b-MO (SEQ ID NO: 3) and cdh5-MO (SEQ ID NO: 4). The standard control-MO served as negative control.

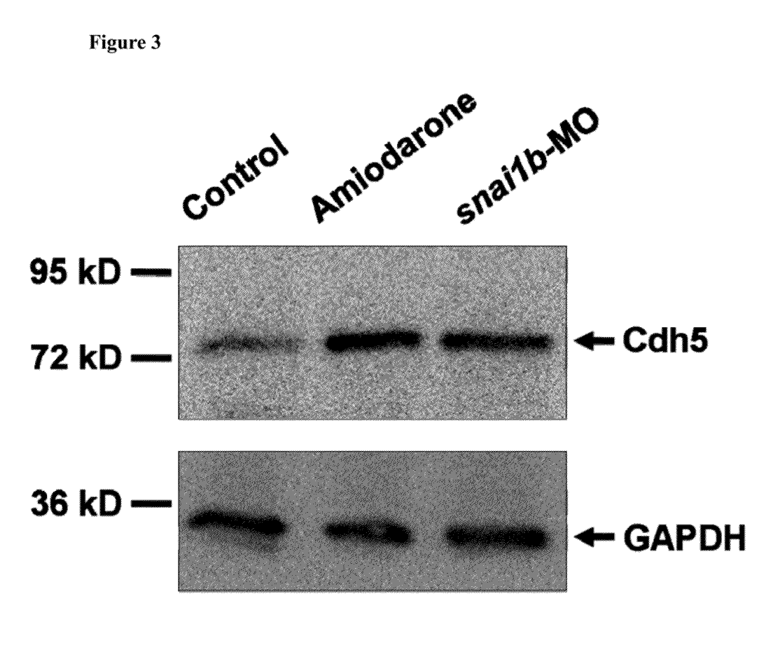

Proteins from 72 hpf zebrafish embryos which were treated with 15 μM Amiodarone from 55 to 72 hpf (lane2) or injected with 1.5 ng snail1b-MO (SEQ ID NO: 3) at one cell staged (lane 3) were collected and the expression level of Cdh5 was analyzed. The expression of Cdh5 protein after Amiodarone treatment was 1.32-fold of that of wild-type. The expression of Cdh5 protein in the one injected with 1.5 ng snail1b-MO (SEQ ID NO: 3) was 1.5-fold of wild-type (FIG. 3). It showed that the expression of Cdh5 protein was increased by Amiodarone treatment and decreased by the inhibition of snai1b gene. Combined the results above, it was confirmed that Amiodarone treatment increased the Cdh5 protein expression level and snail b acted as the role of the receptor of Cdh5 gene and inhibited the formation of Cdh5 protein.

### Example 4

### The Expression Patterns of Versican Family During Zebrafish Embryogenesis

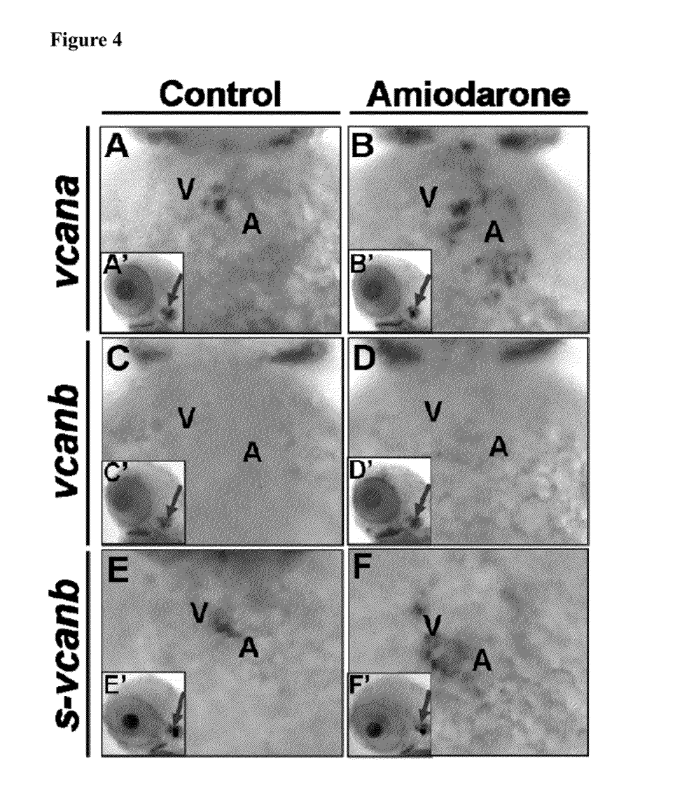

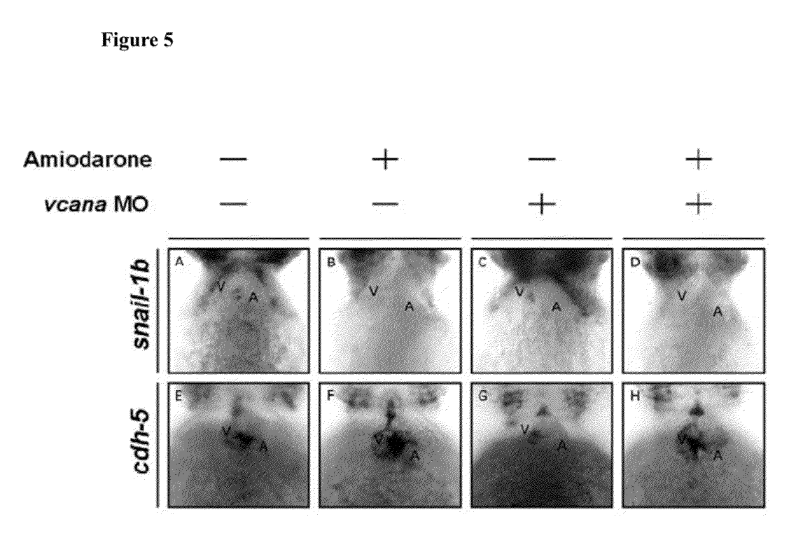

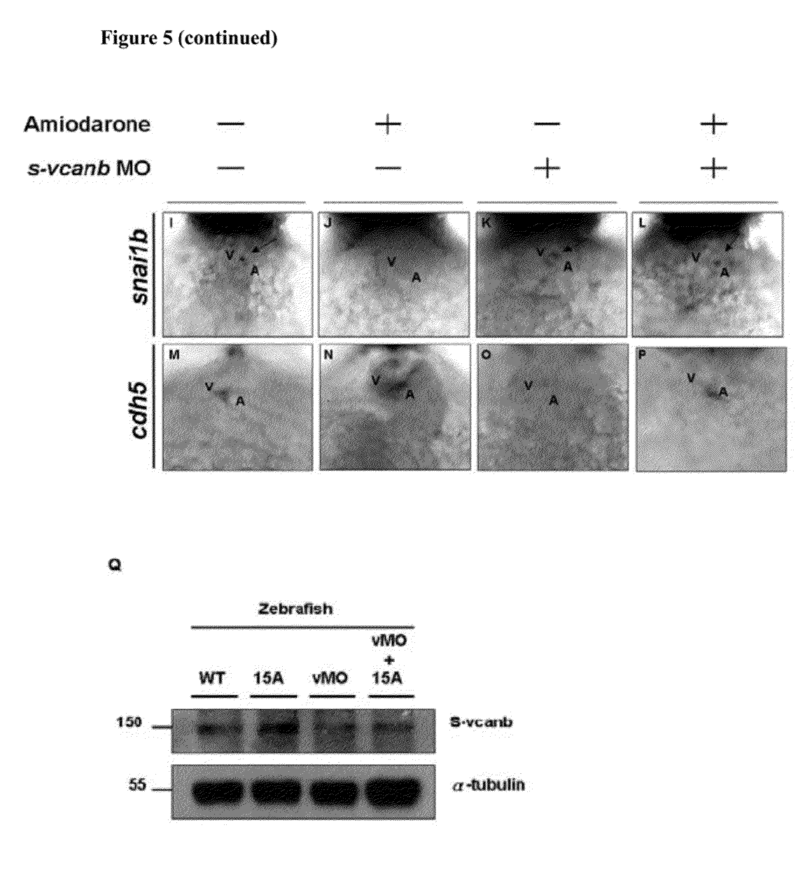

It showed that the ectopic expression of cdh5 was dependent on versican over-expression. In zebrafish, there are three types of versican: Vcana Vcanb and S-Vcanb (Similar to Versican-b). WISH showed that the vcana and s-vcanb were expressed at the AVC during zebrafish cardiac development, but vcanb was not. Thus, it was further focused on vcana and s-vcanb. Although, Amiodarone induced both vcana and s-vcanb at the heart field (FIG. 4B, 4F), it was found that only s-vcanb was involved in Amiodarone inhibiting zebrafish cardiac valve development. Knockdown of vcana did not influence the snail1b (FIG. 5C) and slightly reduced cdh5 transcripts (FIG. 5G) at the heart field. The snail1b were still lost (FIG. 5D) and cdh5 was still ectopic expressed (FIG. 5H) at the heart field in embryos knockdown of vcana and treated with Amiodarone. On the other hand, Knockdown of s-vcanb slightly increased snail1b (FIG. 5K) and reduced cdh5 (FIG. 5O) at the heart field. Knockdown of s-vcanb blocked the effects of Amiodarone: the snail1b was still present (FIG. 5L) and cdh5 was not ectopic expressed (FIG. 5P). Western blots showed that the expression of S-vcanb protein were increased in Amiodarone treated embryos, and reduced in s-vcanb-morphant (FIG. 5Q). The level of S-vcanb was not increased in embryos injected with s-vcanb-MO (SEQ ID NO: 2) and treated with Amiodarone proved that the s-vcanb-MO (SEQ ID NO: 2) used here was specific (FIG. 5Q).

### Example 5

### Amiodarone Influence s-Versican-b/Snail1b/Cdh5 Signaling Through the EGF Motif of s-Versican-b

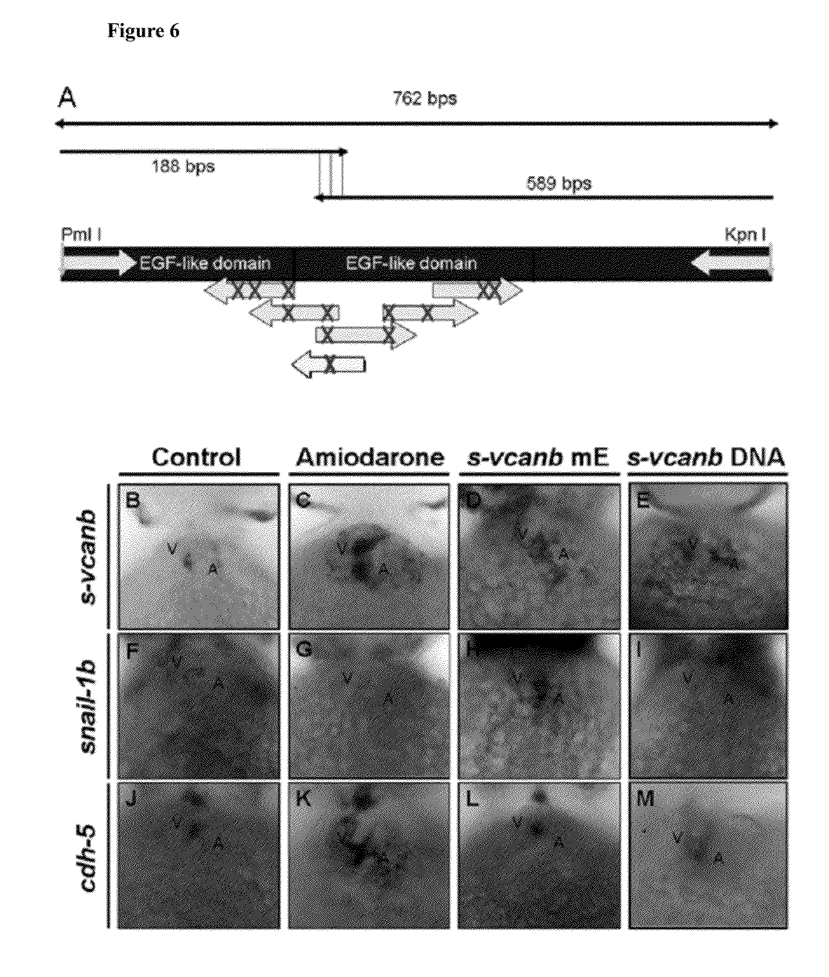

Polymerase chain reaction (PCR)-based in vitro mutagenesis and transgenic assays were used to understand whether the EGF motif of the S-vcanb involved in Amiodarone inhibiting zebrafish cardiac valve development. The zebrafish S-vcanb EGF motif contains 8 defining cysteine residues that form specific disulfide bridges responsible for the secondary structure of the motif. As reported by Schrijver et al., (1999) (Am J Hum Genet. 1999 65:1007-1020), 8 cystenine were mutated to Arginine using 6 primers to disrupt the specific disulfide bridges (FIG. 6A) and termed S-vcanb-mE. Linearlized plasmid DNA containing wild-type S-vcanb or mutant S-vcanb-mE driven by CMV promoter were injected into one cell-staged embryos and analyzed at 72 hpf. Embryos injected with wild-type S-vcanb were observed ectopic s-vcanb signals at the heart field (FIG. 6E vs 6B), suggesting that the injected plasmid DNA were transcribed in the cardiac cells. Similar to the pattern in Amiodarone treated embryos (FIGS. 6C, G and K), the loss of snail1b (FIG. 6I) and ectopic expression of cdh5 (FIG. 6M) in s-vcanb over-expression embryos confirmed that the effects of Amiodarone on cardiac valve were dependent on S-vcanb. Embryos injected with mutated Svcanb-mE were also observed ectopic s-vcanb signals at the heart field (FIG. 6D). The snail1b did not disappear (FIG. 6H) and the cdh5 was still restricted at the AVC (FIG. 6L) in Svcanb-mE over-expressed embryos, indicating that the EGF motif of Svcanb indeed involved in regulating Snail1b and Cdh5.

### Example 6

### Amiodarone Induced Zebrafish s-Vcanb Represses EGFR Signaling in the Heart Field

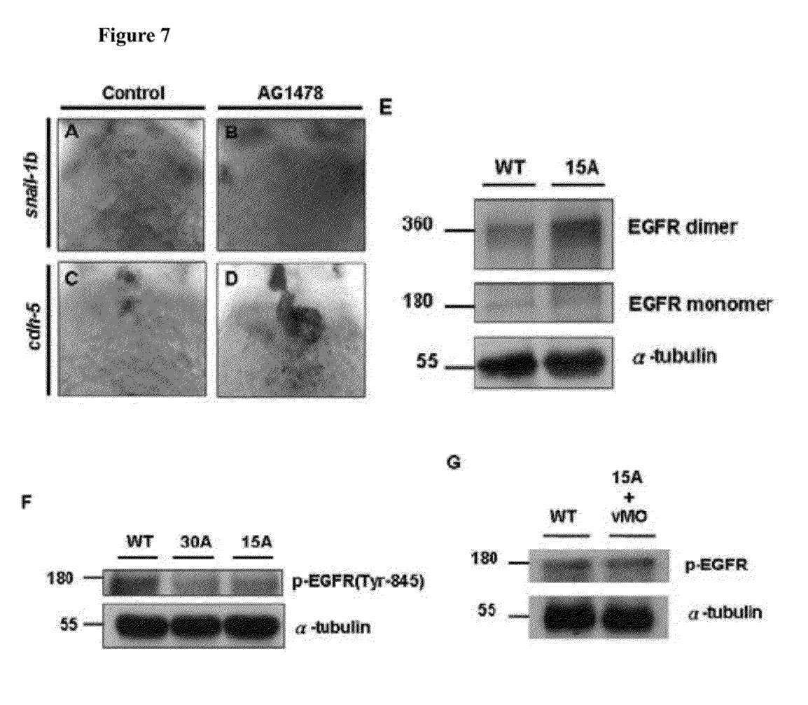

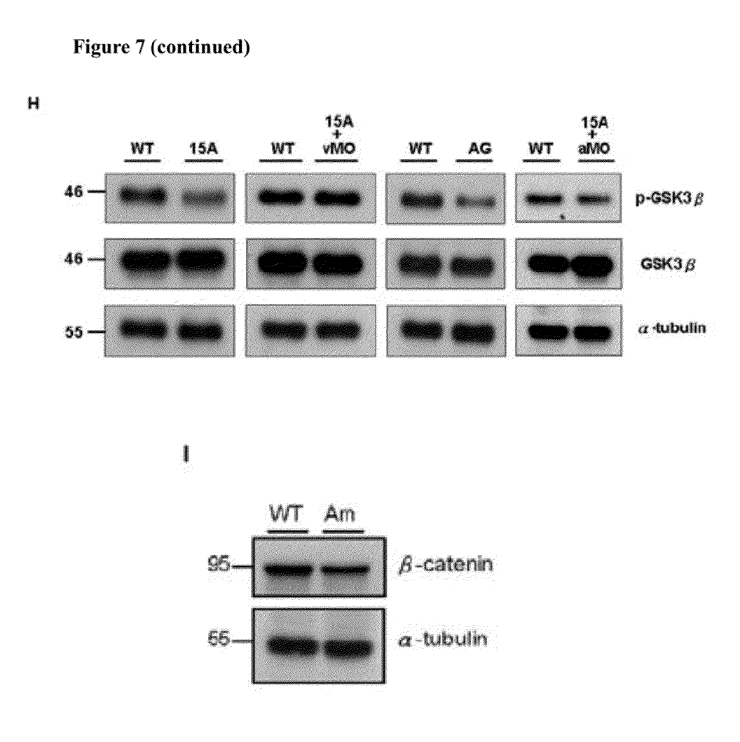

Next, inhibitors were used to test whether Amiodarone induced Svcanb expression influenced EGFR-mediated signaling. Embryos at 55 to 72 hpf were treated with 7 μM of EGFR inhibitor, AG1478. It was found that the snail1b was lost (FIG. 7B). To detail analyze the downstream EGFR signaling, the activity of GSK3β and cdh5 ectopic expression were analyzed (FIG. 7D) at the heart field in AG1478 treated cells. These phenotypes were the same to that of Amiodarone treated embryos and wild-type S-vcanb over-expressed embryos. Examine the oligomeric state of EGFR indicated that Amiodarone treatment increased the EGFR dimeration (FIG. 7E). However, the phosphorylation of EGFR was reduced in Amiodarone treated embryos in a dosage dependent manner (FIG. 7F). Additionally, knockdown of S-vcanb rescued the reduced EGFR phosphorylation caused by Amiodarone (FIG. 7G). These data indicates that the inhibition of EGFR activity by Amiodarone is dependent on Svcanb expression which phosphorylated Snail1b and let it undergo protein degradation. It was found that the total level of GSK3β was unchanged, but the activity of GSK3β was increased significantly in Amiodarone treated embryos and EGFR inhibited embryos (AG1478 treatment; FIG. 7H). Knockdown of S-vcanb blocked the effects of Amiodarone: the activity of GSK3β was not increased. However, knockdown Vcana did not: the activity of GSK3β was still increased. Taken together, the data indicated that Amiodarone induced ectopic Svcanb expression. The ectopic Svcanb then interact with EGFR by its EGF motif to inhibit EGFR signaling. Reduced EGFR signaling results in inhibition of Snail functions through increased GSK3β activity, and thereby cdh5 up-regulation at the heart field.

### Example 7

### 10˜30 μM Amiodarone Did not Influence B16OVA and JC Survival

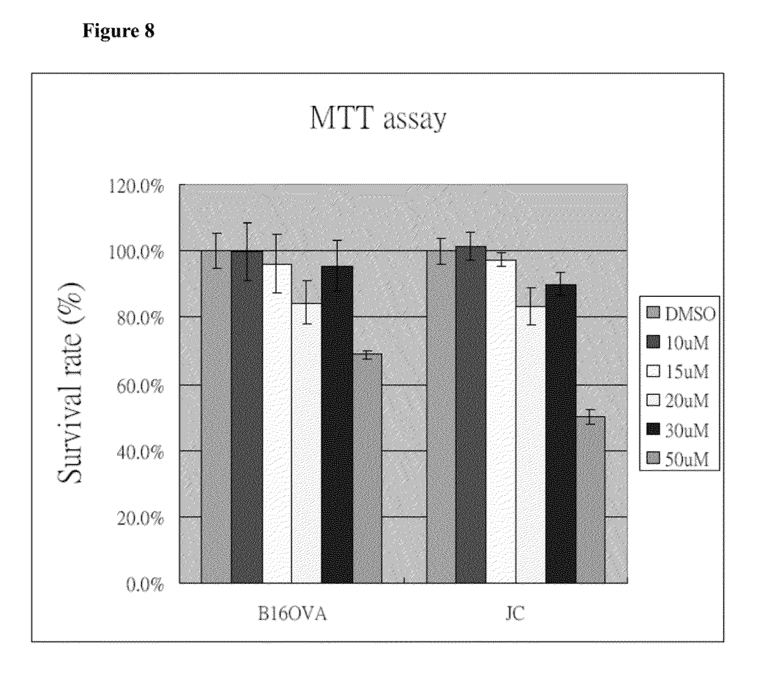

It is well known that the Versican V1 increases EGFR signaling. However, the results revealed that Amiodarone induced zebrafish S-vcanb expression to inhibit EGFR signaling. Interestingly, it was reported that the V2 isoform exhibited opposite biological activities to V1 isoform. Therefore it was proposed that the function of zebrafish S-vcanb conserved to mammal V2 isoform: Amiodarone may induce V2 isoform in mammal cells. In vitro studies have reported that Amiodarone caused cytotoxicity to cells. Thus, firstly, MTT assay were done to confirm that the concentration of Amiodarone used here would not cause cytotoxicity to cells. Cells were plated at a density of 1×10⁴ per well in 96-well plates and incubated for 12-16 h to allow cells to adhere. After 24 h, 0.5 mg/mL 3-(4,5-dimethylthiazol-2-yl)-2,5-diphenyltetrazolium bromide (MTT; Sigma) was added, and the cells were incubated for 4 h. Following incubation, each well was added 100 μl of 10% SDS/0.01N HCl solution and shaking for 5 min. Then, the absorbance was measured at 570 nm. Results were representative of three independent experiments, each done in quintuplicate. It was found that 24 hr of 15 μM Amiodarone treatment did not influence B16OVA (murine B16OVA melanoma cells) and JC cell (JC mouse mammary adenocarcinoma cell line) survival (FIG. 8). Cells were treated with 15 μM Amiodarone in the further experiments.

### Example 8

### Amiodarone Induced Versican V2 Expression and Reduced Snail but Increased E-Cadherin Protein Level

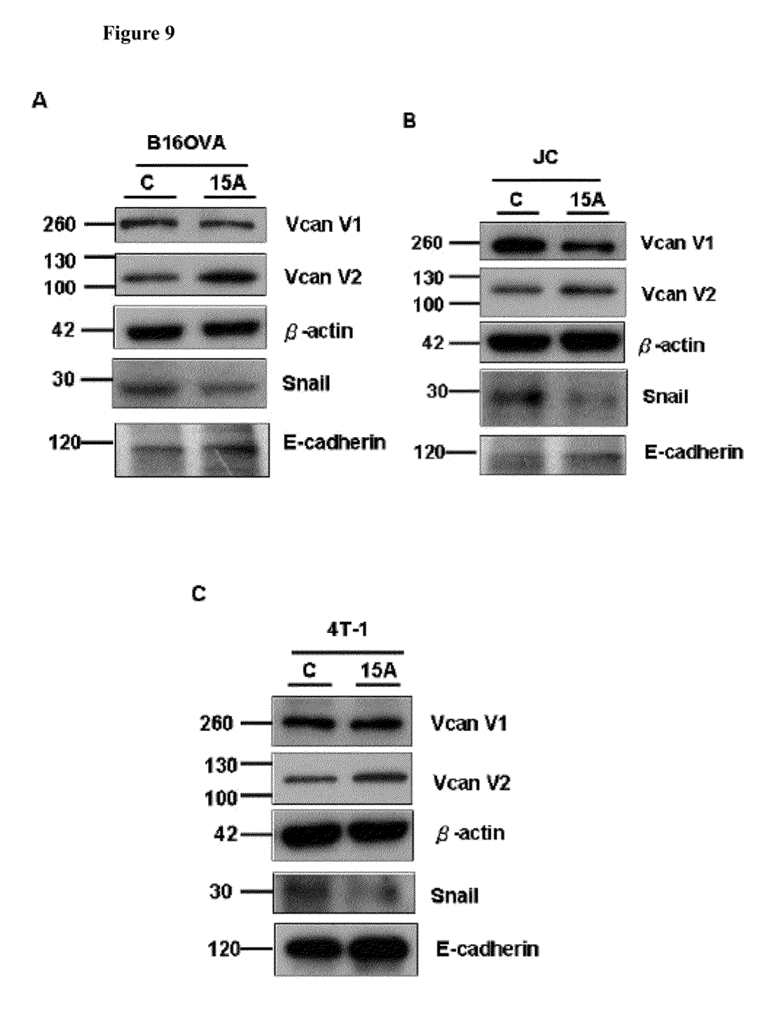

To understand whether Amiodarone induces Versican V2 isoform in mammal cells, the expression of versican V2 isoform and the related molecules were examined in B16OVA (FIG. 9A), JC (FIG. 9B) and 4T-1 cells (mouse mammary carcinoma cell line) (FIG. 9C), which were treated with 15 μM of Amiodarone for 24 hr. All three cells showed increased V2 expression in Amiodarone treated cells than in control cells, indicating that Amiodarone induced V2 expression in mammal system. After Amiodarone treatment, only in B16OVA (FIG. 9A) and JC cells (FIG. 9B) the V1 expression was reduced, but in 4T-1 cells, no difference in V1 expression was shown (FIG. 9C). These indicated that not all the Versican isoform were influenced by Amiodarone. To understand whether the effects of Amiodarone in tumor cells were also similar to that of in zebrafish embryos, the Snail and E-cadherin levels were analysd in Amiodarone treated cells. It was found that the Snail was greatly reduced and E-cadherin was increased significantly in B16OVA (FIG. 9A), JC (FIG. 9B) and 4T-1 cells (FIG. 9C), indicating that Amiodarone functions were conserved in species. Amiodarone induced Versican V2 expression and inhibited Snail expression and finally causeed E-cadherin increased.

### Example 9

### Amiodarone Inhibited Cell Migration

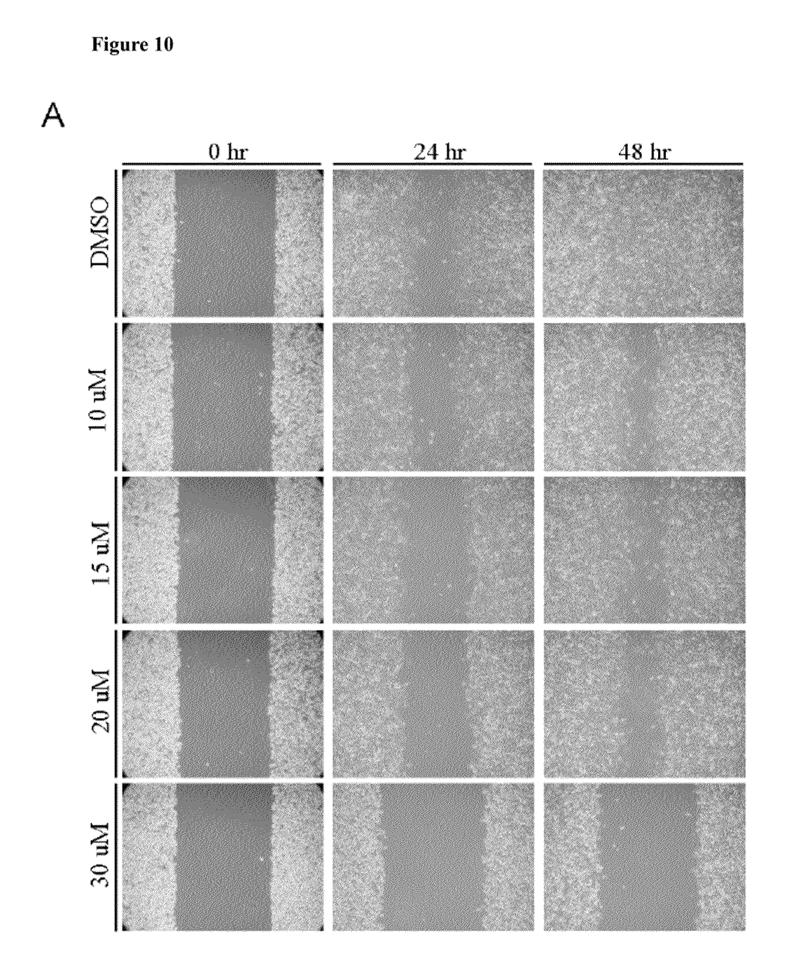

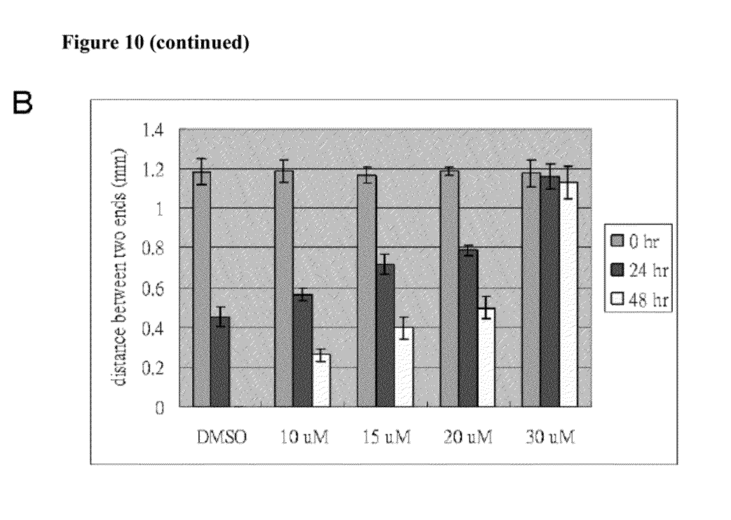

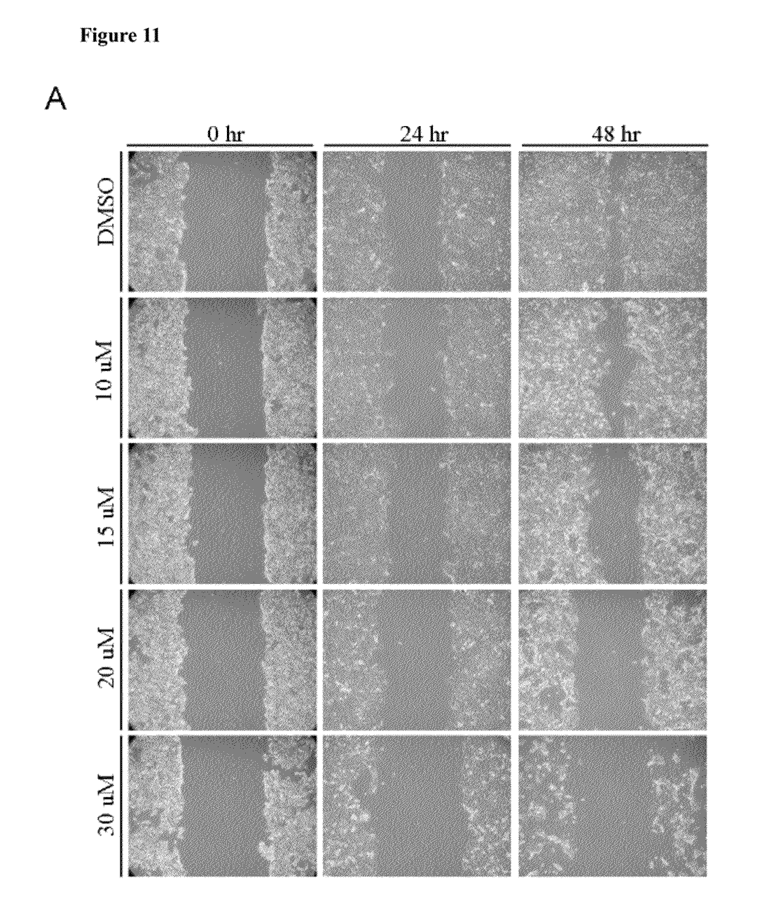

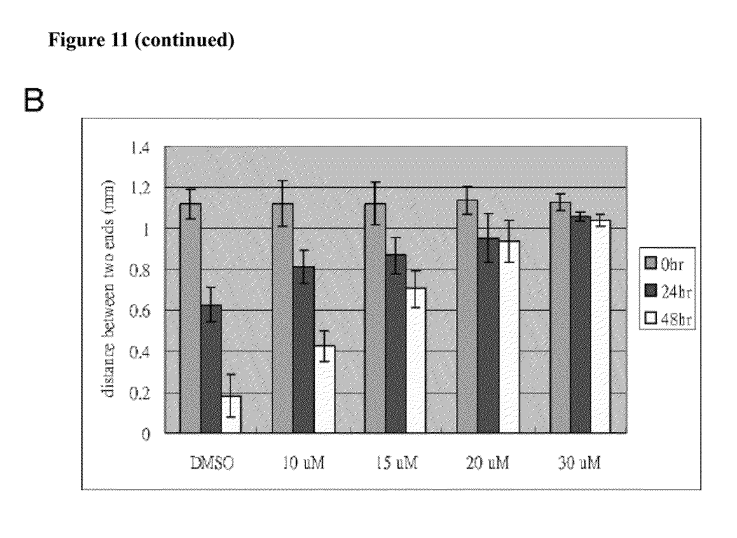

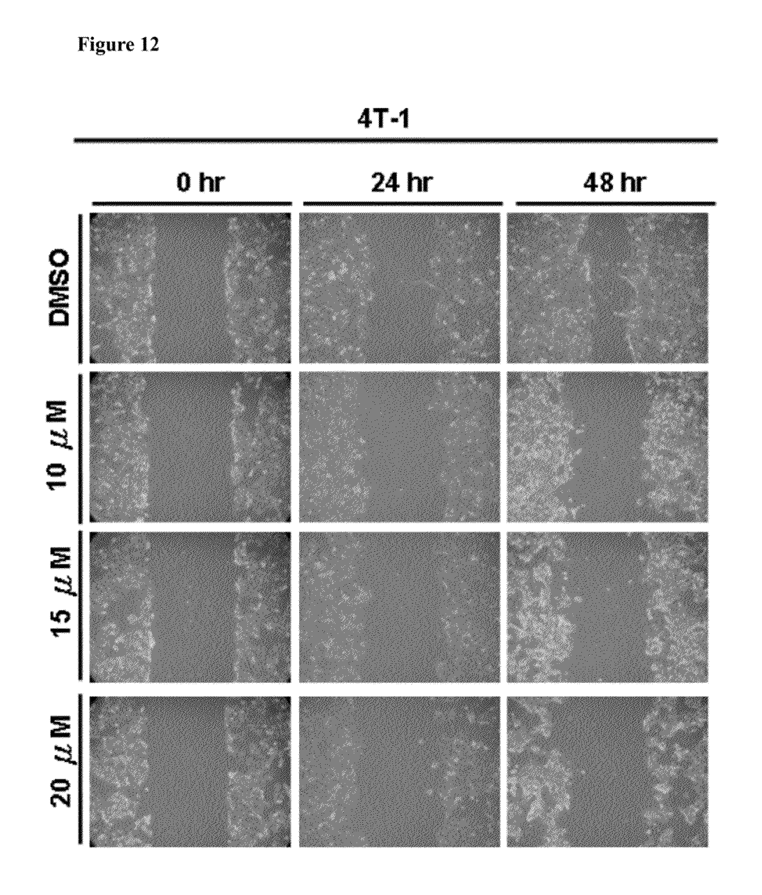

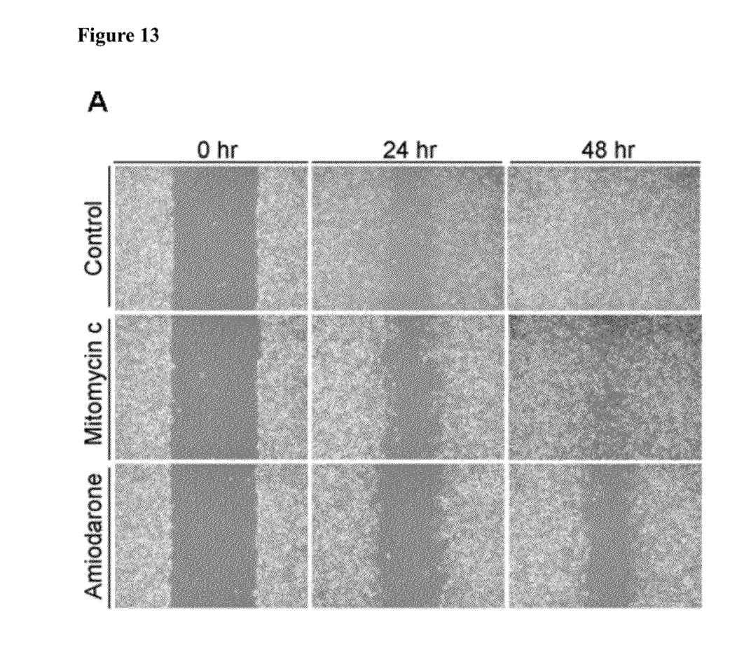

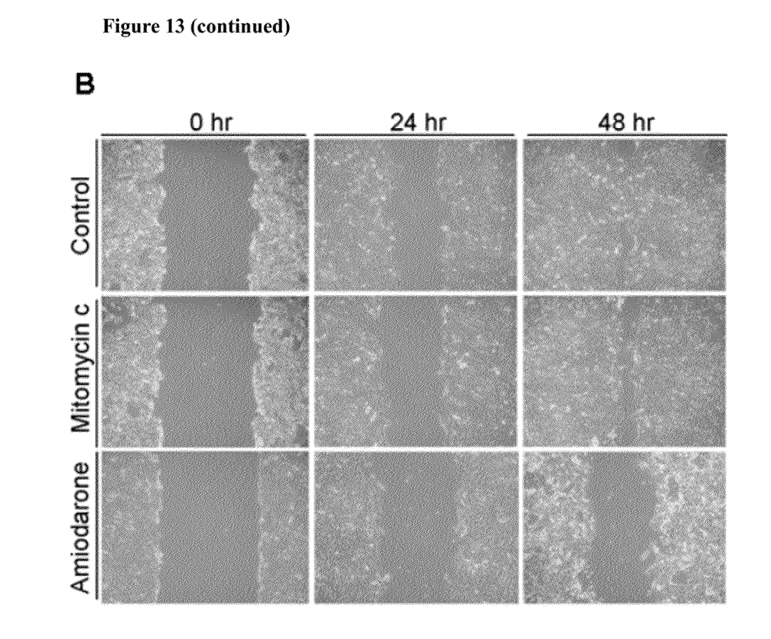

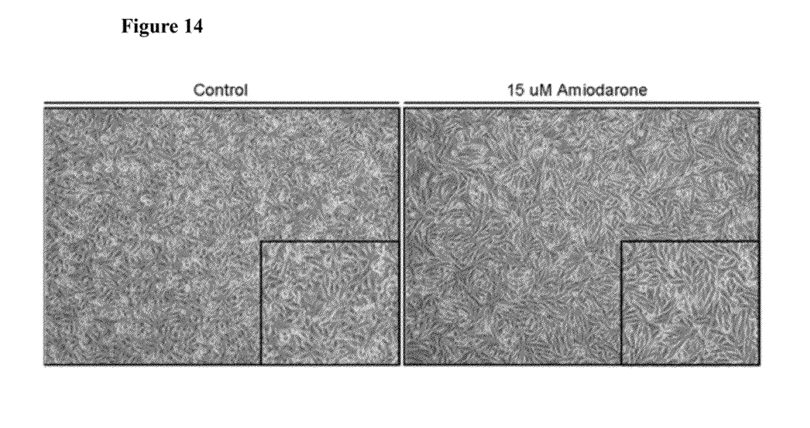

For wound healing assays, B16OVA, JC or 4T-1 cells (1×10⁶) were seeded onto 6-well plates in DMEM/RPMI 1640 medium and maintained at 37° C. until they reached 95% confluence. The monolayer of cells was wounded by a sterile pipette tip to create a 1-mm cell-free path. Culture medium was removed and the samples were washed with PBS, followed by culturing in DMEM/RPMI 1640 medium with 10 μg/ml of the Mitomycin C. Cells were photographed under a low-magnification microscope. As well, the wounded cultures were incubated with medium containing 15 μM Amiodarone, followed by photography. The distances between the wounding centre and the front of the migrating cells (vertical axis) were measured for statistical analysis. In wound healing assays, 24 hr and 48 hr of Amiodarone treated cells all showed lost migratory capacity to the wounding areas, as compared with the control cells (FIGS. 10, 11 and 12). The inhibitory effect of Amiodarone was dependent on its concentration. To determine cell migration in the absence of cell proliferation, Mitomycin C was used to block mitosis and thus allowed the analysis of cell migration in the absence of cell proliferation. Treated with Mitomycin C alone did not affect the time course of wound closure of B16OVA and JC cells (FIG. 13). However, cells treated with both Mitomycin C and Amiodarone did not show significant migration. Additionally, morphological change in Amiodarone treated cells was also found. Amiodarone treated B16OVA cells appeared more stripe and spread out than control cells (FIG. 14), suggesting that Amiodarone treated cells appeared more adhesive than control cells. Taken together, Amiodarone inhibited cell migration in mammal cells through inhibit Snail expression and thereby E-cadherin up-regulated.

### Example 10

### Amiodarone Inhibits Cell Proliferation Through Inhibits EGFR Downstream PI3K/AKT and ERK Signaling

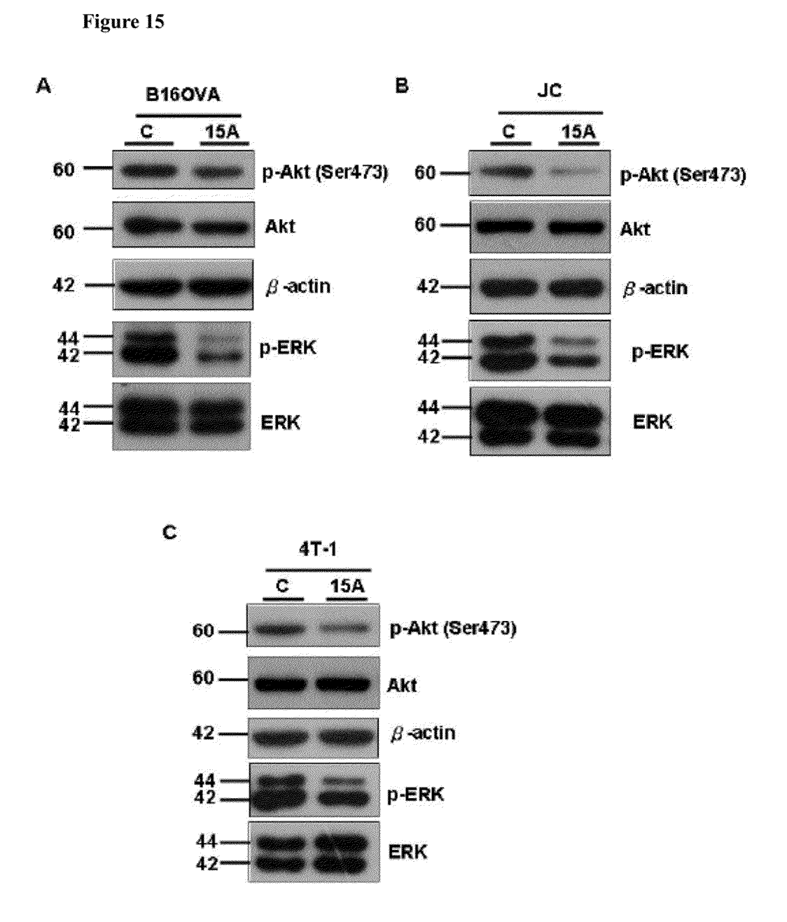

The EGFR signaling regulates cell proliferation through activates EGFR's intrinsic kinase and leads to activation of several downstream intracellular signaling pathways, including rat sarcoma-MAPK kinase (MEK)-extracellular-related kinase (ERK) and phosphoinositide 3-kinase (PI3K)-Akt pathways. It was reported the opposing effects of V1 and V2 on cell proliferation. The possibility that Amiodarone induced Versican V2 affected cell proliferation through regulation of EGFR and its downstream signaling pathway including Akt and the MAP kinases ERK was explored. It was noted that total level of AKT and ERK were unchanged in Amiodarone treated B16OVA (FIG. 15A), JC (FIG. 15B) and 4T-1 cells (FIG. 15C) than in control cells. However, both phosphorylated AKT and phosphorylated ERK42/44 were reduced greatly in Amiodarone treated cells than in control cells. These results indicated that Amiodarone induced Versican V2 was able to regulate EGFR signaling and influence cell proliferation.

### Example 11

### Amiodarone Inhibits Tumor Growth

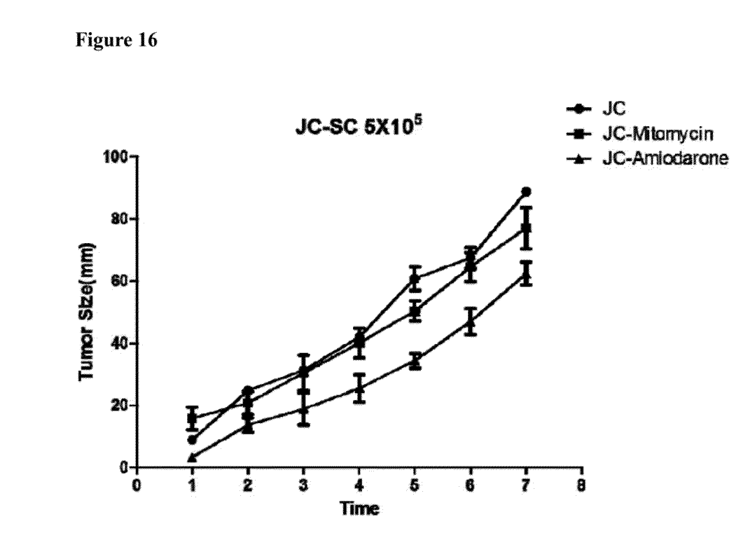

Balb/c mice were inoculated by SC injection into the flank with contorl-Mitomycin C- or Amiodarone-treated JC cells. Each group had 3 mice, which were assigned to experimental groups randomly. All the other mice were sacrificed 2 weeks after treatment. Tumor growth kinetics demonstrated that the Amiodarone treated tumors grew slower than that of the control group and Mitomycin C-treated group (FIG. 16). Thus, it was shown that Amiodarone inhibited tumor growth.

1. A method for inhibiting cancer metastasis in a subject in need thereof comprising administering to the subject a pharmaceutically effective amount of an Amiodarone or its salt.

2. The method of claim 1, wherein the salt is hydrochloride.

3. The method of claim 1, wherein the cancer is selected from cancer cells with an increased expression of Versican V1 or an Amiodarone inducible expression of Versican V2.

4. The method of claim 3, wherein the cancer is selected from cancer cells with an abnormal expression of EGFR.

5. The method of claim 1, wherein the inhibition of cancer metastasis is due to an inhibition of epithelial-mesenchymal transition of the cancer by the Amiodarone.

6. The method of claim 5, wherein the inhibition of epithelial-mesenchymal transition is through an inhibition of EGFR signaling pathway by the Amiodarone.


SEQUENCE LISTING (6 sequences)

SEQ ID NO: 1 - DNA, 25 bp, Artificial
  Designed Morpholino oligonucleotides based on zebrafish vcana nucleotides
aggaagatacccatatttctgctga

SEQ ID NO: 2 - DNA, 25 bp, Artificial
  Designed Morpholino oligonucleotides based on zebrafish s-vcanb nucleotides
ctgaaacacccatgggagtggacat

SEQ ID NO: 3 - DNA, 25 bp, Artificial
  Designed Morpholino oligonucleotides based on zebrafish snai1b nucleotides
ttgacaagaaatgagcgtggcatct

SEQ ID NO: 4 - DNA, 24 bp, Artificial
  Designed Morpholino oligonucleotides based on zebrafish cdh5 nucleotides
tttacaagaccgtctcctttccaa

SEQ ID NO: 5 - DNA, 25 bp, Artificial
  Designed Morpholino oligonucleotides based on zebrafish troponin T2a, cardiac nucleotides
catgtttgctctgatctgacacgca

SEQ ID NO: 6 - DNA, 25 bp, Artificial
  Designed Morpholino oligonucleotides based on standard control nucleotides
cctcttacctcagttacaatttata